(19) United States
(12) Patent Application Publication
(10) Pub. No.: US 2014/0161897 A1
(43) Pub. Date: Jun. 12, 2014

(54) METHOD FOR TREATING CANCERS, SUCH AS MYELOMA AND BREAST CANCER, WITH A COMBINATION OF TBL-12 AND VELCADE

(71) Applicant: Samuel J. GRANT, Port Vila (VU)

(72) Inventor: Samuel J. GRANT, Port Vila (VU)

(21) Appl. No.: 13/709,777

(22) Filed: Dec. 10, 2012

(51) Int. Cl. A61K 35/56; A61K 31/69

(52) U.S. Cl. CPC A61K 35/616; A61K 31/69
USPC 424/520

(57) ABSTRACT

The invention relates to the use of TBL-12 in combination with at least one drug known to be effective against a specific cancer, but at a dose below its minimal effective dose when used alone. Among the types of cancer contemplated for treatment are multiple myeloma, breast, prostate, and cervical cancer. For multiple myeloma, combination with Velcade is envisioned.

### RELATED APPLICATION

This application claims priority from U.S. Provisional Application No. 61/568,748 filed Dec. 9, 2011, incorporated by reference in its entirety.

### FIELD OF THE INVENTION

This invention relates to a method for treating cancer. More particularly, it relates to the treatment of myeloma and breast cancer, via administration of the material referred to as “TBL-12” and elaborated upon infra, either with or without the drug “Velcade.”

### BACKGROUND AND PRIOR ART

Multiple myeloma (“MM” hereafter) is a cancer which evolves from a pathological state known as “monoclonal gammopathy of undetermined significance.” It is characterized by latent accumulation of plasma cells in bone marrow. MM may be described as a malignancy of terminally differentiated B cells, and accounts for approximately 10% of all hematological malignancies.

While some of the symptoms of MM include end organ hypercalcemic diseases, renal insufficiency, anemia, and bone lesions, patients may in fact be asymptomatic, and there is no standard therapy for such patients. While some efficacy has been shown with thalidomide, it is severely toxic.

“Bortezomib” or “Velcade,” is widely used in the treatment of MM, and it has shown to provide remarkable response rates in both relapsed and newly diagnosed MM patients; however, the drug is associated with resistance and toxicity issues.

Current clinical practice in treating MM includes controlling the disease and keeping patients in remission, and to improve life quality via supplementing their therapeutic regime with natural, chemopreventive agents. These include, e.g., resveratrol and curcumin, each of which have been shown to possess anti-tumor properties against myeloma, and promyelocytic leukemia cells, via reducing osteoclast formation, and sensitizing cells which promote multiple mechanisms associated with apoptosis.

Preclinical studies have shown that Velcade enhances the sensitivity of myeloma cells to conventional anti-myeloma agents.

One characteristic of MM is that its progression has been correlated to angiogenesis. Hence, one way to determine efficacy of a substance in MM therapy is to observe its impact on angiogenesis.

“TBL-12” is a natural product, extracted from sea cucumber and other natural, marine ingredients. In parallel experiments, the inventors determined that TBL-12 possesses chemopreventive properties against cancer. In view of these findings, studies were undertaken to deter mine if combinations of TBL-12 and Velcade functioned in a synergistic manner. It has been found that, in fact, the combination of TBL-12 and Velcade has a strong anti-proliferative effect on cancers, such as myeloma. Further, the combination permits the use of doses of Velcade which are below the minimums previously contemplated as being necessary for an anti-cancer effect.

These and other aspects of the invention will be seen in the disclosure which follows.

### BRIEF DESCRIPTION OF THE DRAWINGS

FIG. 1 sets out the results of experiments using a combination of TBL-12 and Velcade on myeloma.

FIG. 2 parallels FIG. 1 in showing the result of the combination on VEGF production in HUVEC cells.

FIG. 3 shows the results of studies on the use of the combination on tube formation in HITVEC and HPEC cells.

### DETAILED DESCRIPTION OF PREFERRED EMBODIMENTS

### Example 1

This example serves as background to what follows, and shows the anti-cancer properties of TBL-12.

Two human myeloma cell lines, which are publicly available (KMS1 and ARP1), were cultured, under standard conditions (96 and 6 well plates in RPMI medium with 10% fetal calf serum). Any cells which reached 75% confluence were used in the experiments which follow.

A stock solution of 1 mg/ml TBL-12, extracted in DMSO, was diluted to produce concentrations of 25, 75, 100, 200, 250, and 500 μg/ml. The myeloma cell lines raised to confluence were then tested, in 96 well plates, with the varying concentrations of TBL-12. A standard MTT assay was carried out to measure cell proliferation. The cells which were cultured in 6 well plates were used in Trypan blue exclusion studies to measure viability.

Examination of cells 48 hours after treatment showed dramatic decreases in cell death. Nuclear condensation in both cell line types showed that apoptotic mechanisms were involved in the cell death. The effect became more pronounced at dosages of 100 μg/ml or higher.

In view of these results, studies were extended to a third multiple myeloma cell line (MM1), a prostate cancer cell line (PC-3), and a human cervical cancer line, “Caski.” This line is also positive for human papilloma virus HPV-16.

While a necrotic effect was not observed, reduced cancer cell proliferation was seen with all cell lines tested.

While not intending to be bound by theory, it is thought that TBL-12 may damage cancer cell membranes which are associated with cell cycle arrest and suppression of cell division.

While LD₅₀ doses differed (100 μg/ml for ARP1, 200 μg/ml KMS1), the effect of TBL-12 was clear.

### Example 2

Different human myeloma cells (U266, MM1, KMS1, and APR1), which are commercially available were cultured in standard 96 well plates using standard methods to exponential growth face. The cells were stimulated by either 1L-6 (5 ng/ml), or TNFα (5 ng/ml).

The cells were then contacted with a combination of a constant concentration of TBL-12 (100 ug/ml), either by itself, or with various concentration of Velcade (2.5, 5, 10, and 15 ng). Cells which received neither TBL-12 nor Velcade were used as a control. Cell survival was measured, using an MTT assay, at 24, 48, and 72 hours.

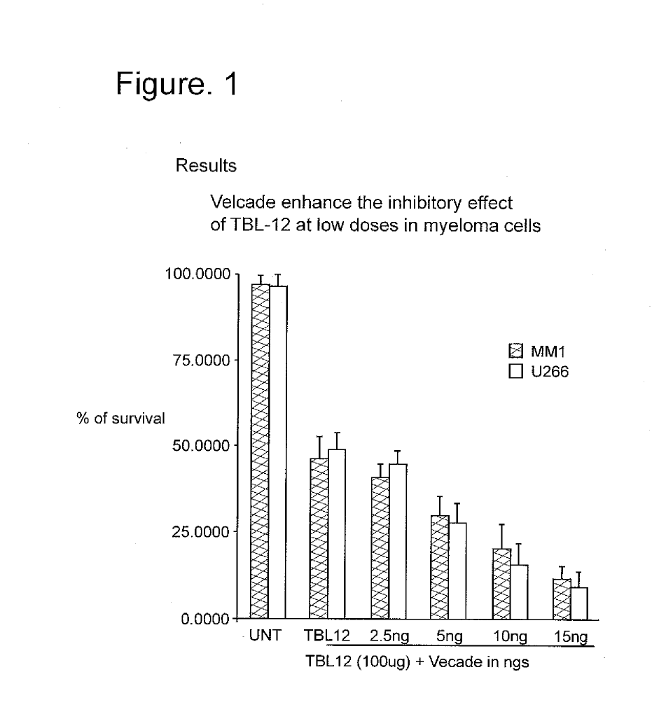

The results, depicted in FIG. 1, showed a clear dose dependent relationship between cell survival and the TBL-12/Velcade “cocktail.” There was efficacy even at the lowest dose of Velcade. The IC₅₀ for Velcade, in combination with the TBL-12, was about 5 ng/ml.

While results are shown for two myeloma cell lines, they are representative of the results for all of the lines tested.

With respect to time dependency, survival dropped from 100% to 30% at 48 hours, and to 20% at 72 hours.

### Example 3

In these experiments, the effect of TBL-12 plus Velcade on the co-culturing of myeloma cells with human umbilical vein endothelial cell (HUVEC) was tested.

Samples of myeloma cells were co-cultured with HUVEC cells, following standard methods, after which a combination of 100 μg/ml TBL-12 and 5 ng/ml of Velcade were added.

Several parameters were measured, the first of which was cell adhesion, i.e., adhesion of the myeloma cells, to the HUVEC cells. This parameter was measured via standard phase contrast and immunofluorescent microscopy, and showed a decrease of 45% in adhesion for cells treated with the drug combination as compared to controls.

Also determined were the levels of VEGF and IL-6 produced in the culture medium. The medium was assayed for VEGF via a standard ELISA, using a commercially available product. Controls were untreated cells, as well as cells treated with TBL-12 only.

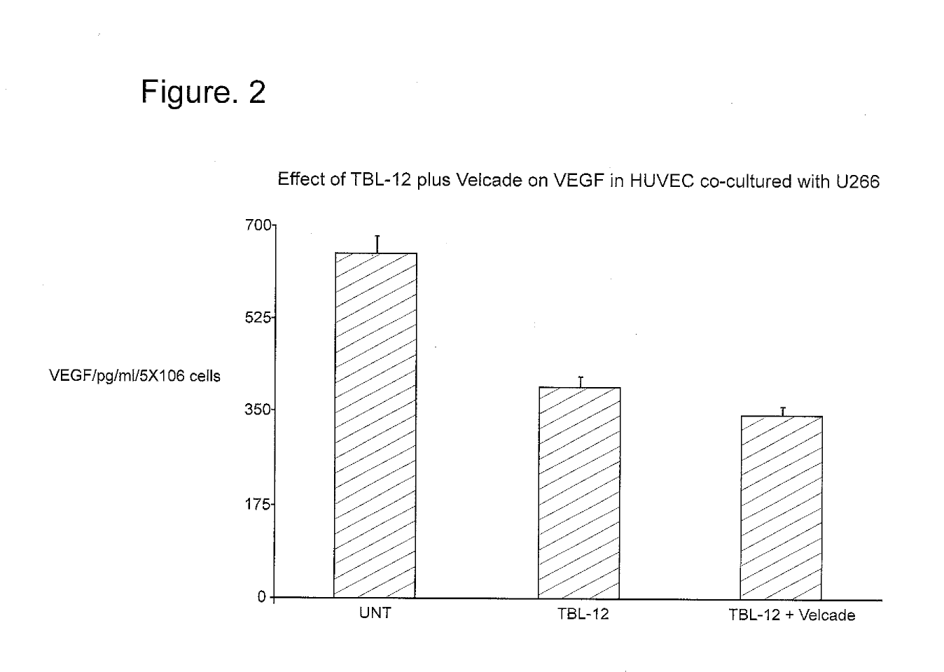

The results, shown in FIG. 2, demonstrate that TBL-12 had a dramatic effect on production of VEGF, and the combination of TBL-12 with Velcade was even better. Similarly, cells were stained for the VEGF receptor known as CD309+VE or VEGFR-2/KDR. Again, there was a drop in expression of the receptor, which was more prominent with the combination of drugs than with TBL-12 alone. These findings suggest that TBL-12's effect was enhanced via a down regulation of IL-6 and TNF-α mediated signaling on VEGF, and VEGFR expression, which in turn suggests inhibition of angiogenesis in myeloma tumor formation.

### Example 4

These experiments were designed to determine the effect of TBL-12 on endothelial tube formation. Endothelial tube formation is a critical feature of the process by which endothelial cells are involved in angiogenesis.

Assaying the formation permits the artisan to determine if angiogenesis is occurring and, if so, to what degree.

HUVEC or HPEC were placed in 96 well plates, coated with Matrigel, a commercially available substrate for facilitating angiogenesis. The aforementioned cells had been treated previously with TBL-12 or Velcade. Either 10 ng/ml VEGF, or 10 ng/ml IL-6. DMSO was the solvent for each of these, and it was used as a control.

The seeded cells were incubated, at 37° C., and tube formation images were observed with a digital, microscope camera system, at different points in time. The number of tubes formed was quantified, via measuring tube length in more than 5 randomly chosen fields.

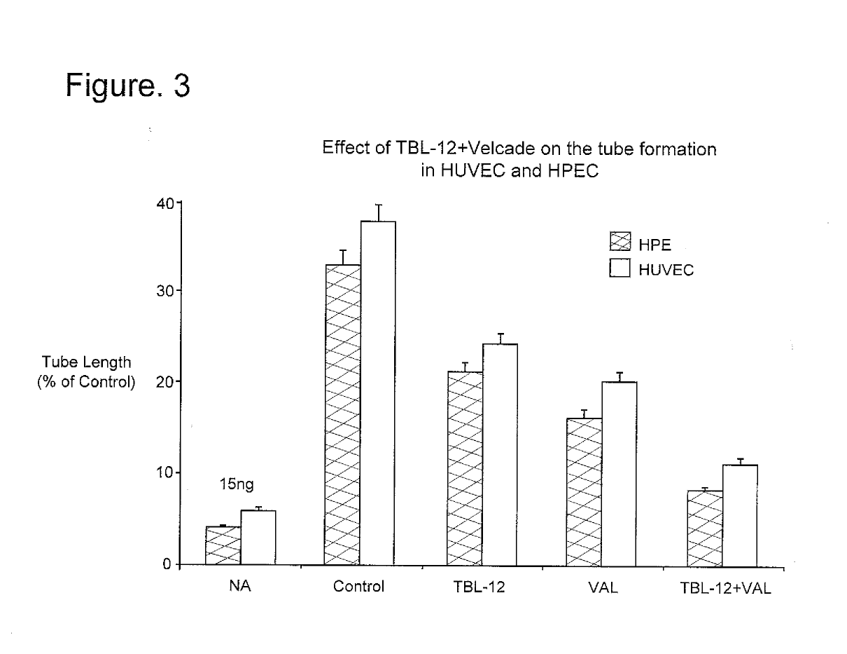

The results shown in FIG. 3, demonstrate a very pronounced improvement when a combination of TBL-12 and Velcade were used, as compared to either drug alone as well as the control, further supporting the hypothesis advance in Example 2, supra.

The studies presented supra are now being repeated for breast cancer, using combinations of TBL-12 and standard, well known breast cancer drugs.

The foregoing examples set forth various features of the inventing, which include a method for treating cancer in a subject, by administering a combination of TBL-12 and Velcade, or another known anti-cancer drug, in an amount sufficient to inhibit proliferation of said cancer.

The cancer treated may be, e.g., myeloma, breast, prostate, or cervical cancer. In the case of myeloma, TBL-12 is administered with Velcade, which has known efficacy against multiple myeloma. The agent will vary in the case of, e.g., breast, prostate, cervical, or other cancers, with the dose of the agent being less than necessary when used alone.

In the case of myeloma, the dose of Velcade may range from about 2.5 ng to about 10 ngs, which is significantly less than the amount used when Velcade is used alone (the standard dose per vial is 3.5 mg). The amount of TBL-12 may range from, e.g., about 25 μg/ml to about 200 μg/ml. Preferably, the dose of Velcade is from about 2.5 ng to about 5.0 ng, and that of TBL-12, from about 100 μg/ml to about 200 μg/ml.

The mode of administration will vary depending upon the severity of the disease and the type of cancer being treated. Preferably, TBL-12 is administered orally, as a food or beverage supplement, while Velcade is administered intravenously via bolus, but the skilled artisan will be familiar with other forms of administration.

The order in which the drugs is administered may vary with one preceding the other or vice versa. They may also be administered “simultaneously,” meaning in this context that there is essentially little or no time in between the administration.

Other features of the invention will be clear to the skilled artisan and need not be reiterated here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1. A method for inhibiting cancer cell growth in a subject in need thereof, comprising administering to said subject an amount of TBL-12 and a drug which is effective against said cancer, said drug being administered in an amount less than the amount effective against said cancer when used alone.

2. The method of claim 1, wherein said cancer is multiple myeloma.

3. The method of claim 2, wherein said drug is Velcade.

4. The method of claim 3, wherein said Velcade is administered at a dose of from about 2.5 ng to about 5.0 ng, and said TBL-12 is administered at a dose of from about 100 mg/ml to about 200 mg/ml.

5. The method of claim 1, wherein said cancer is breast cancer, cervical cancer, or prostate cancer.

6. A kit useful in treating cancer comprising a separate portion of each of (i) TBL-12, and (ii) a drug which is effective against a specific cancer, at a dose below that which is effective for said drug when used alone, (iii) instructions for administering (i) and (ii), and (iv) a container means for holding (i), (ii), and (iii).

7. The kit of claim 6, wherein (ii) is Velcade.

8. The kit of claim 7, wherein said Velcade is present at a dose of from about 2.5 ng to about 5.0 ng, and said TBL-12 is present at a dose of from about 100 mg/ml to about 200 mg/ml.
